(19) United States
(12) Patent Application Publication
(10) Pub. No.: US 2025/0185958 A1
(43) Pub. Date: Jun. 12, 2025

(54) Device and Method for Detecting Enzymatic Activity

(71) Applicants: Stichting IMEC Nederland, Wageningen (NL); Stichting Radboud Universiteit, Nijmegen (NL)

(72) Inventors: Arjan Tibbe, Deventer (NL); Rachel Armstrong, Eindhoven (NL); Christian Büll, Bemmel (NL); Thomas Jan Boltje, Doorn (NL)

(21) Appl. No.: 18/973,874

(22) Filed: Dec. 9, 2024

(30) Foreign Application Priority Data
Dec. 11, 2023 (EP) 23215454.2
Nov. 21, 2024 (EP) 24214439.2

(51) Int. Cl. A61B 5/1486; A61B 5/145; A61B 5/1459

(52) U.S. Cl. CPC A61B 5/14865; A61B 5/14507; A61B 5/14532; A61B 5/1459

(57) ABSTRACT

A device is provided for detecting enzymatic activity in mucus layer. The device comprises at least one mucin molecule corresponding to an enzyme of a first type, wherein the mucin molecule is coupled to a surface of the device and is configured to be in contact with mucus layer contents, at least one probe coupled to an end of the mucin molecule opposite to the surface of the device, and a detector configured to detect a presence of the probe and/or a property of the probe in order to detect a status of the mucin molecule.

### CROSS-REFERENCE TO RELATED APPLICATIONS

The present application is a non-provisional patent application claiming priority to European Patent Application No. EP 23215454.2, filed Dec. 11, 2023, and European Patent Application No. EP 24214439.2, filed Nov. 21, 2024, the contents of each of which are hereby incorporated by reference.

### FIELD OF THE DISCLOSURE

The present disclosure relates to the detection of enzymatic activity in the mucus layer that lines the walls of the body's organs and especially the mucus layer of the gastrointestinal (GI) tract, the respiratory tract and the reproductive organs.

### BACKGROUND

The mucus layer may be regarded as the first line of defense against the infiltration of microorganisms. A healthy and intact mucus layer is essential to prevent colonization and invasion by pathogens. A healthy intact mucus layer contains a healthy population of commensal bacteria that maintain a symbiotic relationship with the host. By measuring the presence of enzymes produced by the commensal mucus residing bacteria, the type of bacteria and, thereby, the health status of the mucus layer may be identified.

At present, protease activity detection schemes generally relate to ex situ measurements performed on a cultured molecule in a controlled environment. For example, Ji Ji et al. (Electrochemical detection of the activities of thrombin and its inhibitor, Electrochemistry Communications, Volume 16, Issue 1, 2012, Pages 53-56, doi: 10.1016/j.elecom.2012.01.004) discloses the detection of thrombin activities in a controlled environment. However, such a detection scheme cannot work when measuring biomarkers in complex matrices like the GI-tract.

Accordingly, an object of the present disclosure is to provide devices and methods to facilitate in vivo measurements, especially within the mucus layer of the GI-tract, to measure the presence of enzymes produced by commensal bacteria residing in the mucus layer.

### SUMMARY

In a first aspect, the present disclosure provides a device for detecting enzymatic activity in mucus layer. The device includes at least one mucin molecule corresponding to an enzyme of a first type. The mucin molecule is coupled to a surface of the device and is configured to be in contact with mucus layer contents. The device further includes at least one probe coupled to an end of the mucin molecule opposite to the surface of the device and a detector configured to detect a presence of the probe and/or a property of the probe in order to detect a status of the mucin molecule.

In one embodiment of the first aspect, the mucin molecule includes engineered human TR-O glycodomains and/or mucin-like domains decorated with O-glycans.

In one embodiment of the first aspect, the mucin molecule is configured to be digested by the enzyme of the first type produced by the commensal microbes that reside in the mucus layer.

In one embodiment of the first aspect, the probe is an optical probe and the detector is configured to detect an optical signal from the probe, optionally, wherein the optical probe is a fluorescent probe and the detector is configured to detect a fluorescence of the fluorescent probe.

In one embodiment of the first aspect, the probe is a redox probe and the detector is configured to detect an oxidation-reduction activity of the redox probe.

In one embodiment of the first aspect, the device further includes a linker molecule configured to couple the mucin molecule to the surface of the device. The linker molecule is inert with respect to the mucus layer contents and other gastrointestinal, GI, tract contents.

In one embodiment of the first aspect, the device further includes: at least one further mucin molecule corresponding to an enzyme of a second type, wherein the at least one further mucin molecule is coupled to the surface of the device and is configured to be in contact with the mucus layer contents; and at least one further probe coupled to an end of the at least one further mucin molecule opposite to the surface of the device, wherein the detector is configured to detect a presence of the further probe and/or a property of the further probe in order to detect a status of the at least one further mucin molecule. In one embodiment, the at least one further mucin molecule is configured to be digested by the enzyme of the second type produced by commensal microbes that reside in the mucus layer. In one embodiment, the detector is configured to detect the presence of the probe and the presence of the further probe simultaneously, or wherein the detector is configured to detect the presence of the probe and the presence of the further probe sequentially, and/or wherein the detector is configured to detect the property of the probe and the property of the further probe simultaneously, or wherein the detector is configured to detect the property of the probe and the property of the further probe sequentially.

In some embodiments, the mucin molecule and the at least one further mucin molecule are coupled to the surface of the device at predefined locations, optionally in an array formation. In some embodiments, the status of the mucin molecule or the at least one further mucin molecule is the enzymatic degradation and/or removal of the mucin molecule or the at least one further mucin molecule.

In a second aspect, the present disclosure provides a method for detecting enzymatic activity in mucus layer. The method includes the steps of coupling at least one mucin molecule corresponding to an enzyme of a first type to a surface of a device such that the mucin molecule is in contact with mucus layer contents, coupling at least one probe to an end of the mucin molecule opposite to the surface of the device, and detecting a presence of the probe and/or a property of the probe in order to detect a status of the mucin molecule.

In one embodiment of the second aspect, the method further comprises: coupling at least one further mucin molecule corresponding to an enzyme of a second type to the surface of the device such that the at least one further mucin molecule is in contact with the mucus layer contents, coupling at least one further probe to an end of the at least one further mucin molecule opposite to the surface of the device, and detecting a presence of the further probe and/or a property of the further probe in order to detect a status of the at least one further mucin molecule.

In one embodiment of the second aspect, the method further comprises: detecting the presence of the probe and the presence of the further probe simultaneously, or detecting the presence of the probe and the presence of the further probe sequentially, and/or detecting the property of the probe and the property of the further probe simultaneously, or detecting the property of the probe and the property of the further probe sequentially.

In one embodiment, the status of the mucin molecule or the at least one further mucin molecule is the enzymatic degradation and/or removal of the mucin molecule or the further mucin molecule.

In a third aspect, the present disclosure provides a device for detecting enzymatic activity in a mucus layer. The device includes a mucin molecule coupled to a surface of the device by a linker, wherein the mucin molecule is configured to be in contact with mucus layer contents; at least one probe coupled to an end of the mucin molecule opposite to the surface of the device; a detector configured to detect a signal from the probe; and a transmitter configured to receive the signal from the probe and transmit the signal to an external device.

In one embodiment of the third aspect, the mucin molecule comprises engineered human TR-O glycodomains or mucin-like domains decorated with O-glycans.

In one embodiment of the third aspect, the mucin molecule is digestible by a glycosidase, a sulphatase, and/or a sialidase within the mucus layer.

In one embodiment of the third aspect, the probe is a fluorescent probe and the detector is a fluorescence detector.

In one embodiment of the third aspect, the probe is a redox probe and the detector is configured to detect an oxidation-reduction potential of the probe.

### BRIEF DESCRIPTION OF THE FIGURES

The disclosure above, as well as additional features, will be better understood

through the following illustrative and non-limiting detailed description of example embodiments, with reference to the appended drawings.

FIG. 1 shows a first exemplary embodiment of the device according to the first aspect of the present disclosure;

FIG. 2 shows an exemplary degradation process of the mucin molecule;

FIG. 3 shows a first exemplary embodiment of the detector;

FIG. 4 shows a second exemplary embodiment of the detector;

FIG. 5 shows a second exemplary embodiment of the device according to the first aspect of the present disclosure;

FIG. 6 shows a third exemplary embodiment of the device according to the first

aspect of the present disclosure;

FIG. 7 shows an exemplary enzymatic activity as a function of time; and

FIG. 8 shows an exemplary embodiment of the method according to the second aspect of the present disclosure.

All the figures are schematic, not necessarily to scale, and generally only show

parts which are necessary to elucidate example embodiments, wherein other parts may be omitted or merely suggested.

### DETAILED DESCRIPTION

According to a first aspect of the present disclosure, a device is provided for detecting enzymatic activity in the mucus layer. The device comprises at least one mucin molecule corresponding to an enzyme of a first type, wherein the mucin molecule is coupled to a surface of the device and is configured to be in contact with mucus layer contents, at least one probe coupled to an end of the mucin molecule opposite to the surface of the device, and a detector configured to detect a presence of the probe and/or a property of the probe in order to detect a status of the mucin molecule.

In the present patent application, “corresponding to an enzyme” means digestible/cleavable/degradable by an enzyme and/or digested/cleaved/degraded by an enzyme.

In some embodiments, the at least one mucin molecule coupled to the surface of the device is engineered to correspond to, i.e. to be digestible/cleavable/degradable by a specific type of enzyme.

The device may be an ingestible device, where the detector or sensor may be arranged in an ingestible structure or packaging, such as an ingestible sensing pill. Alternatively, the device may be an implantable device, or a device that can be in physical contact with the mucus layer contents

For the ingestible device, the enzyme-specific mucin molecule may be coupled to a surface of the ingestible structure, especially to an exterior surface of the ingestible structure, so that the mucin molecule is in contact with the contents of the mucus layer, i.e. the sample matrix, especially within the GI-tract, whereby the probe may be coupled to the end of the mucin molecule, e.g., to the top side, opposite to the surface of the ingestible structure.

Accordingly, the detector or sensor may detect or sense a presence of the probe in order to detect a status of the mucin molecule. Additionally or alternatively, the detector may detect or sense or measure a property of the probe in order to detect a status of the mucin molecule.

The status of the mucin molecule according to the present disclosure is defined herein as its glycosylation content and/or extent of glycosylation and/or alterations in glycosylation pattern and/or degradation and/or removal, and/or presence or absence, which offers precise measurements of enzyme presence and activity.

“Enzyme of a first type” is defined as one specific enzyme, such as glycosidases, sulphatases, or sialidases.

“Enzyme of a second type” is defined as one specific enzyme, such as

glycosidases, sulphatases, or sialidases.

In various examples, the enzyme of the first type is different from the enzyme of the second type.

Enzymes produced by commensal microbes residing in the mucus layer, including but not limited to glycosidases, sulphatases, or sialidases, are able to digest glycan structures of the mucin molecule. Degradation and/or removal and/or alteration of the glycosylation and/or glycosylation pattern releases the mucin molecule backbone and renders it accessible to proteases, e.g. glycoproteases, serine proteases, metalloproteases, or mucinases, which in turn digest (degrade and/or remove) the mucin molecule. Degradation and/or removal of the mucin molecule releases (removes) the probe from the sensor surface and results in a detectable alteration of the signal and/or signal intensity level.

In various example embodiments, the alteration of the signal and/or signal intensity level is detected. In some examples, the alteration of the signal and/or signal intensity level is a disappearance of the signal and/or a decrease of the signal intensity level.

In case the enzyme for digesting the glycan structure is not present, the mucin molecule backbone remains protected from the proteases and the probe remains attached to the mucin molecule and, thereby, to the sensor surface. In this case, the signal and/or signal intensity remains at the unchanged, i.e. at the starting level.

Thus, in some embodiments, the presence of enzymes produced by the commensal mucus residing bacteria can be detected so that the type of bacteria and, thereby, the health status of the mucus layer can be identified.

A healthy and intact mucus layer contains a healthy population of commensal bacteria maintaining a symbiotic relationship with the host. The mucus layer serves as a carbon and energy source for mucus residing commensal bacteria, predominantly in the form of glycans. The mucus layer residing commensal bacteria produce mucus-degrading enzymes such as glycosidase, sulphatase, and sialidase cleaving the mucus network to enhance utilization as an energy source to produce monosaccharides utilized by the mucus residing bacteria. In turn, the mucus residing commensal bacterial produce short-chain fatty acids to maintain a functional mucus layer barrier.

The mucus layer is composed of mucins consisting of a large family of heavily O-glycosylated proteins. Mucins are primarily defined by their variable tandem repeat (TR) domains that are densely decorated with different O-glycan structures in different patterns.

Human TR-O-glycodomains consist of approximately 200 amino acids and are small parts of the mucins that can be produced by genetically engineered human HEK293 cells using the method according to Nason et al., Nat Commun 12, 4070 (2021), Display of the human mucinome with defined O-glycans by gene engineered cells.

In some embodiments, the mucin molecule comprises tandem repeat (TR) domains that defines O-glycan binding sites. Alternatively, the mucin molecule may comprise mucin-like domains decorated with O-glycans. For instance, the TR sequence defines the density of O-glycans. The O-glycan structures can be composed of natural, engineered or modified sugar groups hereby creating enzymatic selectivity or blocking enzymatic digesting.

The term mucin molecule according to the present disclosure may also comprise mucin-like molecules. Mucin-like molecules are glycoproteins that contain tandem repeat domains resembling the serine and threonine rich regions of mucin tandem repeat domains with dense mucin-type O-glycosylation. Examples of mucin-like glycoproteins carrying mucin O-glycan domains include but are not limited to CD34, CD43, CD44, CD68, or PSGL1.

In some examples, the mucin molecule is configured to be digested by the enzyme of the first type, for example, those produced by the commensal microbes that reside in the mucus layer. For instance, the mucin molecule may be configured to be digested exclusively by the enzyme of the first type. In this regard, the O-glycan groups that cover the mucin backbone (amino acid backbone) may be configured to be digested exclusively by the enzyme of the first type.

Alternatively, the O-glycan groups may be configured to be digested by a combination of enzymes wherein the enzyme of the first type may digest the first sugar group of the glycan domain and the next available sugar group may be digested by an enzyme of a second type. In some example embodiments, the activity of a specific enzyme or a combination of enzymes produced by the commensal bacteria residing in the mucus layer can be detected.

Further alternatively, the mucin molecule may be configured such that one or more particular enzymes, such as the enzyme of the first type, may not digest or degrade the mucin molecule. In various embodiments, the indigestible or undegradable mucin molecules may serve as stable controls for reference measurements.

In some examples, precision-engineered mucin molecules with specific glycosylation structures that correspond to particular enzymes are applied.

This differs from e.g., using mixtures of mucin molecules with unknown structures and glycosylation profiles.

The present disclosure establishes a one-to-one relationship between engineered mucin molecules and the specific enzyme capable of cleaving the glycan structures from these mucins.

For example, in the case the mucin molecule is configured to be digested by the enzyme of the first type, only one type of enzyme can cleave these glycan structures from the mucin molecules, enabling the detection of both the presence and enzymatic activity of the targeted enzymes.

In various example embodiments, the probe is an optical probe, and the detector is configured to detect an optical signal from the probe. In a preferred embodiment, the optical probe is a fluorescent probe, and the detector is configured to detect fluorescence of the fluorescent probe, especially as the property of the probe. In another preferred embodiment, the optical probe may be an optical absorption probe.

In a further preferred embodiment, the probe is a redox probe, and the detector is configured to detect an oxidation-reduction activity of the redox probe, especially as the property of the probe. For instance, the detector may detect an oxidation-reduction potential of the redox probe. In some examples, a fast detection time and a high detection sensitivity are achieved.

In the case of a fluorescence-based sensor, the presence and activity of an enzyme are detected as a reduction in fluorescence light intensity. No interaction between fluorescent probes and the surface is required.

With a redox label, there must be an electrochemical interaction between the label and the surface to which the mucin is attached.

Generally, redox probes function similarly to fluorescent labels in that they are synthesized at the terminal end of the mucins. Upon enzymatic degradation of the mucins, the redox label is released. When the redox label is no longer attached, the resulting change in electrochemical interaction is the observed signal.

The redox probe in combination with electrochemical detection, may offer various benefits or differences over a fluorescent probe and fluorescence sensing. For instance, fluorescence biosensors generally occupy more space and are more complex in technical realisation. Electrochemical probes facilitate improved miniaturization and reduced power consumption.

In various embodiments, the device comprises a linker molecule configured to couple the mucin molecule to the surface of the device, the linker molecule being inert to the mucus layer contents, especially to the enzymes produced by the commensal microbes that reside in the mucus layer.

This ensures that the loss of signal is only caused by enzymatic mucin degradation.

Furthermore, this may allow attaching synthetic mucins to the surface of the device.

For example, in the case of an ingestible device, the linker molecule may be inert to the enzymes present in the GI-tract. For instance, enzymatic activity may be monitored by measuring the enzymatic digestion of the mucin molecule. The enzymatic activity may lead to a decrease in the number sensor probes and as such loss of signal. The linker molecule is not cleavable by the enzymes or removed by other gastrointestinal contents, ensuring that the loss of signal is only caused by enzymatic mucin degradation.

In various embodiments, nonspecific binding is not measured by the sensor. Thereby, false positive enzymatic protein digestion registration can be prevented, and the detection accuracy can be enhanced.

The linker molecule may be any linker molecule used in the art, such as synthetic amino acid linkers, disulfide linkers, hydrazone linkers. Additionally or alternatively, the linker molecule may comprise or be protein-substrate combinations such as streptavidin-biotin and avidin-biotin, silane molecules containing linkable functional groups like alkanes, primary amines, carboxylic acids, N-hydroxysuccinimde esters, azides, alkynes and so on.

In some examples, the device comprises at least one further mucin molecule corresponding to an enzyme of a second type, wherein the further mucin molecule is coupled to the surface of the device and is configured to be in contact with the mucus layer contents, especially in contact with enzymes produced by the commensal bacteria that reside in the mucus layer, and at least one further probe coupled to an end of the further mucin molecule opposite to the surface of the device.

In this regard, the detector is configured to detect a presence of the further probe and/or a property of the further probe in order to detect a status of the further mucin molecule. In some embodiments, the presence of different enzymes produced by the commensal mucus residing bacteria can be detected.

In various examples, the further mucin molecule is configured to be digested by the enzyme of the second type, for example, those produced by commensal microbes that reside in the mucus layer. For instance, the further mucin molecule may be configured to be digested exclusively by the enzyme of the second type. In this regard, the O-glycan groups that cover the mucin backbone (amino acid backbone) may be configured to be digested exclusively by the enzyme of the second type.

Alternatively, the O-glycan groups may be configured to be digested by a combination of enzymes wherein the enzyme of the first type may digest the first sugar group of the glycan domain and the next available sugar group may be digested by an enzyme of a second type.

In example embodiments, the activity of different enzymes in the mucus layer, especially of enzymes produced by the commensal bacteria residing the mucus layer, can be specifically detected, thereby obtaining an enzymatic signature, e.g., the distribution of enzymes along the mucus layer, e.g. within the GI-tract.

In some examples, the detector is configured to detect the presence of the probe and the presence of the further probe simultaneously. Additionally or alternatively, the detector is configured to detect the property of the probe and the property of the further probe simultaneously. In such scenarios, a parallel detection of enzymatic activity can be facilitated.

In various embodiments, the detector is configured to detect the presence of the probe and the presence of the further probe sequentially. Additionally or alternatively, the detector is configured to detect the property of the probe and the property of the further probe sequentially. In some examples, the accuracy of the enzymatic activity detection can be further enhanced.

In various embodiments, the mucin molecule and the further mucin molecule are coupled to the surface of the device at predefined locations, especially in an array formation. In some examples, the presence of different enzymes can be detected in a simplified manner based on the locations of the different mucin molecules in the array.

In various examples, the status of the mucin molecule or the further mucin molecule is the enzymatic degradation and/or removal of the mucin molecule or the further mucin molecule.

In the present disclosure, a single sensor surface equipped with various mucin molecules is provided. In this setup, various mucin molecules are positioned at predefined locations or specific regions on the sensor surface. The type of mucin is identified based on its specific location on the sensor. A loss of fluorescence signal at a certain location indicates the enzymatic activity of enzymes that cleave the mucins present at that location. This design allows for the simultaneous detection of the presence and activity of multiple enzymes using a single sensor surface.

According to a second aspect of the present disclosure, a method is provided for detecting enzymatic activity in mucus layer. The method comprises the steps of coupling at least one mucin molecule corresponding to an enzyme of a first type to a surface of a device such that the mucin molecule is in contact with mucus layer contents, coupling at least one probe to an end of the mucin molecule opposite to the surface of the device, and detecting a presence of the probe and/or a property of the probe in order to detect a status of the mucin molecule.

In various embodiments, the method further comprises the steps of coupling at least one further mucin molecule corresponding to an enzyme of a second type to the surface of the device such that the further mucin molecule is in contact with the mucus layer contents, coupling at least one further probe to an end of the further mucin molecule opposite to the surface of the device, and detecting a presence of the further probe and/or a property of the further probe in order to detect a status of the further mucin molecule.

In some examples, the method further comprises the step of detecting the presence of the probe and the presence of the further probe simultaneously. Additionally or alternatively, the method comprises the step of detecting the property of the probe and the property of the further probe simultaneously.

In various examples, the method further comprises the step of detecting the presence of the probe and the presence of the further probe sequentially. Additionally or alternatively, the method comprises the step of detecting the property of the probe and the property of the further probe sequentially.

In some embodiments, the status of the mucin molecule or the further mucin molecule is the enzymatic degradation and/or removal of the mucin molecule or the further mucin molecule.

It is to be noted that the method according to the second aspect corresponds to the device according to the first aspect and its implementation forms. Accordingly, the method of the second aspect may have corresponding implementation forms. Further, the method of the second aspect achieves the same characteristics and effects as the device of the first aspect and its respective implementation forms.

Reference will now be made in detail to the embodiments of the present disclosure, examples of which are illustrated in the accompanying drawings. However, the following embodiments of the present disclosure may be variously modified and the range of the present disclosure is not limited by the following embodiments.

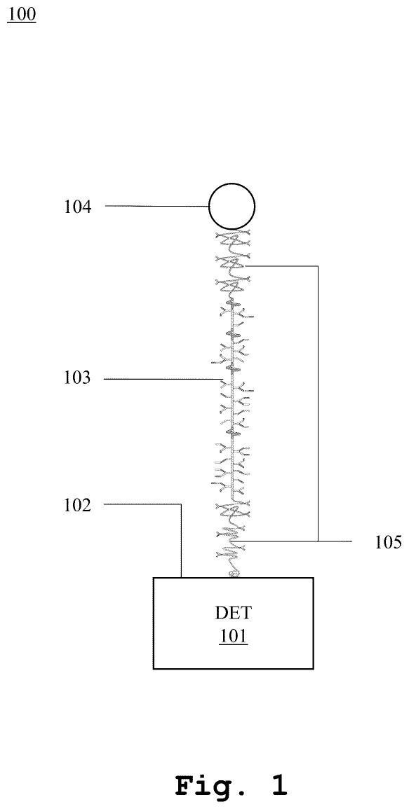

In FIG. 1, a first exemplary embodiment of the device 100 according to one embodiment of the present disclosure is illustrated. Although the device 100 is illustrated as an ingestible device, the device 100 can also be an implantable device. The device 100 may comprise a detector or sensor 101 arranged in an ingestible structure, e.g., a pill, or an implantable structure, such that an external surface 102 of the structure, i.e., a detector or sensor surface, can be accessible for the detection or sensing.

The device 100 may further comprise a mucin molecule 103 of a first type, especially with a known amino acid sequence and structure. As such, the mucin molecule 103 may be configured to be digested primarily by an enzyme of a first type produced in the mucus layer. The mucin molecule 103 may be attached to the detector surface 102 so that the mucin molecule 103 is in contact with the enzymes produced by the commensal bacteria that reside in the mucus layer, e.g., are present in the GI-tract contents.

The device 100 may further comprise a probe or probe molecule 104 attached to an end of the mucin molecule 103, especially to the end of the mucin molecule 103 opposite to the detector surface 102.

In this regard, the mucin molecule 103 may be attached to the detector surface 102 through a linker molecule 105. Additionally, the probe 104 may be attached to the mucin molecule 103 in a similar manner, i.e., through a further linker molecule 105. The linker molecule 105 may be inert to the contents of the mucus layer, e.g., the contents of the GI-tract, especially to the enzymes produced in the GI-tract.

In some embodiments, the probe 104 may be a fluorescent probe and the detector 101 is configured to measure a fluorescence of the probe 104. Alternatively, the probe 104 may be a redox probe and the detector 101 is configured to measure an oxidation-reduction potential (ORP) of the probe 104. As such, a reduction in the measured fluorescence or in the measured ORP may indicate the status, in particular the degradation or removal of the mucin molecule 103, i.e., the mucin protein, thereby detecting an enzymatic activity in the mucus layer, e.g., within the GI-tract.

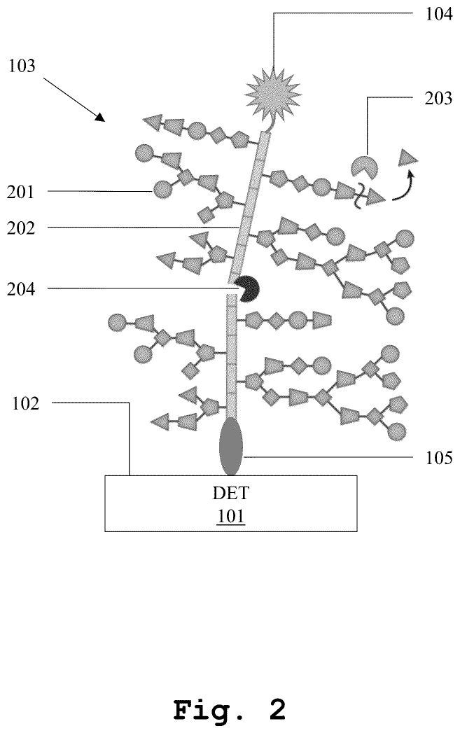

In FIG. 2, an exemplary degradation process of the mucin molecule 103 is illustrated.

Generally, the mucus barrier layer is composed of mucins consisting of a large family of heavily O-glycosylated proteins and constituting the major macromolecules in most body fluids. The mucins can be primarily defined by their variable tandem repeat (TR) domains that are densely decorated with different O-glycan structures in distinct patterns.

For example, human TR-O glycodomains having approximately 200 amino acids may be small parts of the mucin molecules that can be produced by genetically engineered human HEK293 cells.

In particular, the mucus layer may serve as a carbon and energy source for mucus residing commensal bacteria, predominantly in the form of glycans. As an adaptation to residing in a glycan-rich environment, these bacteria produce mucus-degrading enzymes such as glycosidase, sulphatase, and sialidases, which can cleave the mucus network to enhance the utilization of mucus as an energy source. These bacterial species can cleave mucus O-glycans to produce monosaccharides utilized by the mucus-residing bacteria.

In return, these bacteria produce short-chain fatty acids (SCFAs) such as butyrate to regulate intestinal homeostasis. SCFAs may be regarded as an energy source for the epithelial cells and help maintain a functional barrier.

Generally, Inflammatory Bowel Disease (IBD) patients may display a reduction of commensal bacteria, including SCFA-producing bacteria, resulting in mucosal layer damage and leading to a dysfunctional barrier. This results in bacterial penetration, thereby triggering an inflammatory cascade against the invading bacteria. As such, by measuring the presence of enzymes produced by the commensal mucus-residing bacteria, the type of bacteria and the health status of the mucus layer can be determined.

Turning back to FIG. 2, the mucin molecule 103 may comprise O-glycans 201 covering the protein backbone 202. In the presence of O-glycan-degrading enzymes 203, such as glycosidase, sulphatase, or sialidase, the O-glycan sugar groups 201 may be digested one by one by the O-glycan-degrading enzymes 203 and the protein backbone 202 may become exposed to proteases 204 that are present inside the sample matrix. In case the enzymes 203, 204 digest the mucin molecule 103, i.e. the status of the mucin molecule changes, the probe 104 may be released, especially from the detector surface 102, which may result in the disappearance and/or reduction of the measured fluorescence or of the measured ORP.

As such, the digestion process illustrated in FIG. 2 can be regarded as a two-step process:
1. The digestion of the O-glycans 201 on the mucin molecule 103 by the enzymes 203 produced by the commensal bacteria that reside in the mucus layer, and
2. After the removal of sufficient O-glycans, the amino acid backbone 202 of the mucin molecule 103 may become exposed to the “general available” proteases 204, which may result in a break, i.e. degradation, of the mucin molecule 103 and therefore a release (removal) of the probe 104.

It is to be noted that the linker molecule 105 of the present disclosure may not be cleavable by the enzymes 203 and the proteases 204, especially to avoid false positive measurements of protein degradation. In a further aspect, the sample matrix may not contain enzymes capable of cleaving the linker molecule.

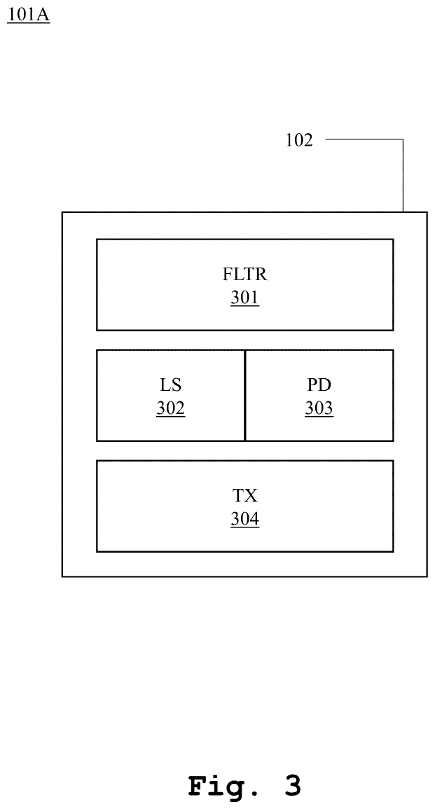

In FIG. 3, a first exemplary embodiment of a detector 101A is illustrated. The detector 101A may be a fluorescence detector and may detect the fluorescence of the probe 104.

For example, the detector 101A may comprise a filter or grating structure (FLTR) 301 configured for an excitation light signal of a predefined wavelength and further for an emission light signal of a predefined wavelength. For instance, the filter structure 301 may be arranged at or near the detector surface 102.

The detector 101A may further comprise a light source (LS) 302, such as a light-emitting diode, which may generate the excitation light signal to illuminate the probe 104 especially through the filter structure 301, e.g., to achieve the excited state of the probe molecule.

The detector 101A may further comprise a photodetector (PD) 303, such as a photodiode, which may receive the fluorescent emission or the emission light signal from the probe 104 or may detect the fluorescent emission or the emission light signal of the probe 104, especially through the filter structure 301. The fluorescent emission may result from the transition of the probe molecule from the excited state to the reference or ground state.

The detector 101A may further comprise a transmitter (TX) 304 that may receive the fluorescent measurements from the photodetector 303, e.g., the photodiode currents or voltages, and may transmit the measurements to an external device, especially wirelessly, for further pursing and/or processing of the measurement data.

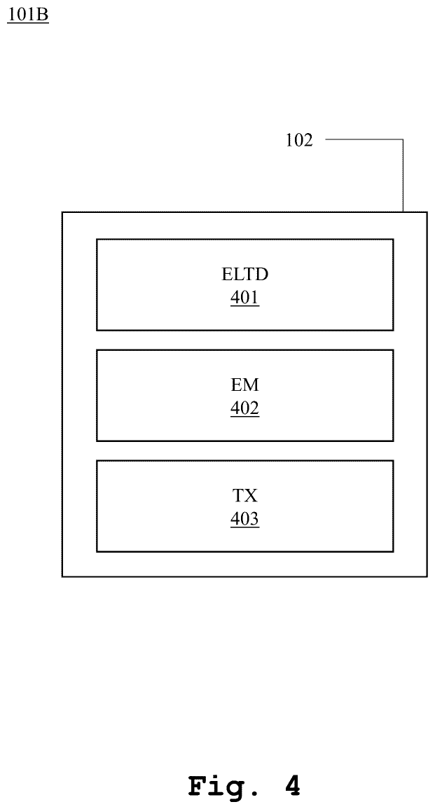

In FIG. 4, a second exemplary embodiment of the detector 101B is illustrated. The detector 101B may be an ORP detector and may detect or measure the ORP of the probe 104.

For example, the detector 101B may comprise an electrode (ELTD) 401 configured to be electrically coupled to the probe 104. For instance, the electrode 401 may be arranged at or near the detector surface 102.

The detector 101B may further comprise an electrometer (EM) 402, such as a potentiometer, electrically coupled to the electrode 401. The electrometer 402 may measure an ORP of the probe 104.

The detector 101B may further comprise a transmitter 403 that may receive the ORP measurements from the electrometer 402 and may transmit the measurements to an external device, especially wirelessly, for further pursing and/or processing of the measurement data.

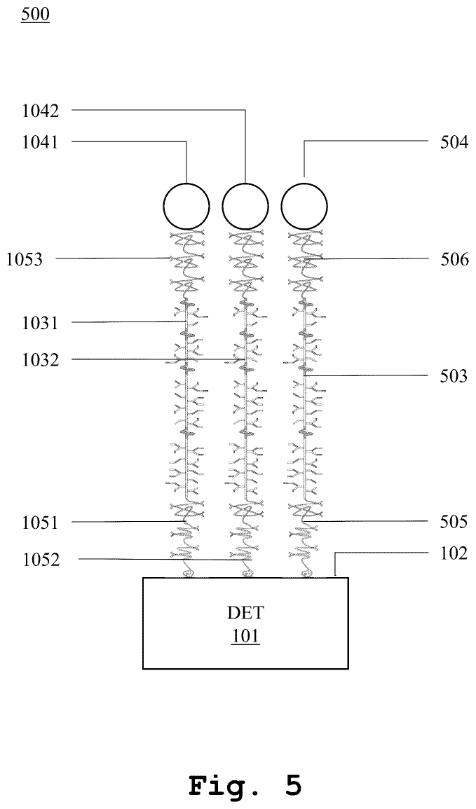

In FIG. 5, a second exemplary embodiment of the device 500 according to the first aspect of the present disclosure is illustrated. The device 500 differs from the device 100 in that the device 500 comprises a plurality of mucin molecules of a first type, for example, two mucin molecules of the first type 1031, 1032, to form a cluster or array of mucin molecules of the first type.

In this regard, each of the mucin molecules 1031, 1032 may be configured to be digested primarily by the enzyme of the first type produced in the mucus layer. Furthermore, each of the mucin molecules 1031, 1032 may be attached to the detector surface 102 through a respective linker molecule 1051, 1052. Moreover, a respective probe 1041, 1042, may be attached to an end of the mucin molecules 1031, 1032, especially to the end of the mucin molecules 1031, 1032 opposite to the detector surface 102, e.g., through a further respective linker molecule 1053.

As such, the cluster of mucin molecules of the first type may facilitate multiple levels of intensity of the measured fluorescence or ORP, i.e., multiple intensity levels or multiple potential levels, which may allow for measuring the rate of protein degradation.

The device 500 further differs from the device 100 in that the device 500 may comprise a mucin molecule 503 of a second type, especially with known amino acid sequence and structure. As such, the mucin molecule 503 may be configured to be digested primarily by an enzyme of a second type produced in the mucus layer.

Furthermore, the mucin molecule 503 may be attached to the detector surface 102 through a further linker molecule 505, which may be similar or the same as the linker molecules 1051, 1052 of the mucin molecule of the first type. Moreover, a further probe 504 may be attached to an end of the mucin molecule 503, especially to the end of the mucin molecule 503 opposite to the detector surface 102, e.g., through a further linker molecule 506.

The probe 504 of the mucin molecule 503 may correspond to the probes 1041, 1042 of the mucin molecules 1031, 1032. Alternatively, the probe 504 of the mucin molecule 503 and the probes 1041, 1042 of the mucin molecules 1031, 1032 may be different probes.

For example, the probe 504 of the mucin molecule 503 may be a fluorescent probe and the probes 1041, 1042 of the mucin molecules 1031, 1032 may be redox probes, or vice versa. Moreover, the device 500 may comprise additional mucin molecules of the second type, e.g., in a cluster or array, in an analogous manner to the cluster of the mucin molecules of the first type, especially to allow for measuring the rate of protein degradation due to the presence of different enzymes.

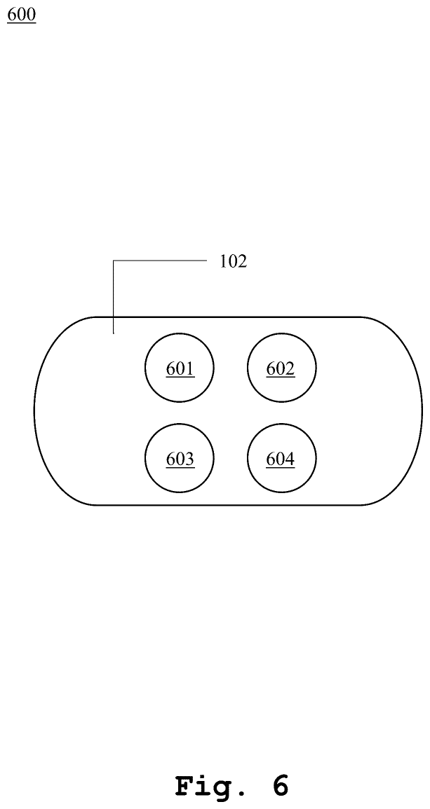

In FIG. 6, a third exemplary embodiment of the device 600 according to the first aspect of the present disclosure is illustrated. In this regard, the device 600, especially the ingestible structure, is illustrated in a pill shape. For example, the device 600 may comprise, especially at the external surface 102, a first location 601, a second location 602, a third location 603, and a fourth location 604.

For example, a cluster of mucin molecules of a first type, such as the mucin molecules 1031, 1032 of FIG. 5, may be arranged in the first location 601. The mucin molecules of the first type may be configured to be digested primarily by an enzyme of a first type produced in the mucus layer.

In addition, a cluster of mucin molecules of a second type, such as the mucin molecules 1031, 1032 of FIG. 5, may be arranged in the second location 602. The mucin molecules of the second type may be configured to be digested primarily by an enzyme of a second type produced in the mucus layer.

Furthermore, a cluster of mucin molecules of a third type, such as the mucin molecules 1031, 1032 of FIG. 5, may be arranged in the third location 603. The mucin molecules of the third type may be configured to be digested primarily by an enzyme of a third type produced in the mucus layer.

Moreover, a cluster of mucin molecules of a fourth type, such as the mucin molecules 1031, 1032 of FIG. 5, may be arranged in the fourth location 603. The mucin molecules of the fourth type may be configured to be digested primarily by an enzyme of a fourth type produced in the mucus layer.

The detector 101 (not shown) may be arranged inside of the ingestible structure such that the detector 101 may operably detect or measure the signals from the probes via the detector surface 102. In this regard, the detector 101 may simultaneously detect or measure the signals from each of the locations 601, 602, 603, 604. Alternatively, the detector 101 may sequentially detect or measure the signals from the locations 601, 602, 603, 604, e.g., in a time-multiplexed manner.

It should be understood that the number of locations illustrated here is exemplary and can be reduced to a smaller number or can be extended to a larger number.

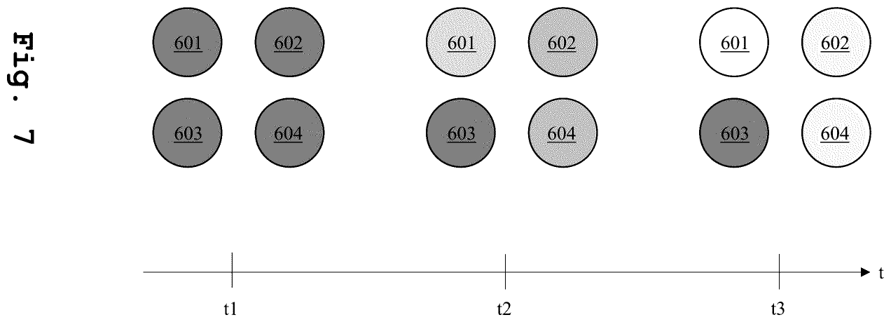

In FIG. 7, an exemplary enzymatic activity on the device 600 is illustrated as a function of time. As discussed before, the locations 601, 602, 603, 604 may indicate the type of glycodomains and the associated signal intensity level of the respective locations 601, 602, 603, 604 may indicate the enzymatic activity corresponding to the type of glycodomains. In FIG. 7, the signal intensity level is illustrated in grayscale where higher signal intensity is shown in darker shades and lower intensity is shown in lighter shades.

For example, at the start at t1, the signal intensity levels at the locations 601, 602, 603, 604 are all shown at maximum due to the absence of enzymatic digestion of the mucins. The signal intensity drops or reduces over time as the enzymes produced by the commensal bacteria may digest the O-glycans, thereby exposing the protein backbone to proteases. The proteases digest (degrade) the mucin molecule(s) resulting in the release, i.e. removal, of the probe, which leads to an alteration of the signal and/or intensity level of the signal. Thus, the mucin molecule status, in particular its degradation and/or removal, is detected by the detector.

Due to the presence of different enzymes at different locations within the mucus layer, e.g., within the GI-tract, the signal intensity at different locations 601, 602, 603, 604 may vary with respect to each other.

In this example, throughout the time instances t2 and t3, the signal intensity at the first location 601 is most reduced, the signal intensities at the second location 602 and the fourth location 604 are moderately reduced, and the signal intensity at the third location 603 is unchanged.

It can therefore be understood, for the third location 603, that the enzyme, which is able to digest this particular mucin, may not be present at all, e.g., throughout the whole GI-tract. Furthermore, the different intensity levels at different locations 601, 602, 603, 604 may correspond to the presence and activity of different glycodomain-specific enzymes, which may allow to assess or envisage the status of the mucus layer, e.g., the mucus layer lining the wall of the GI-tract.

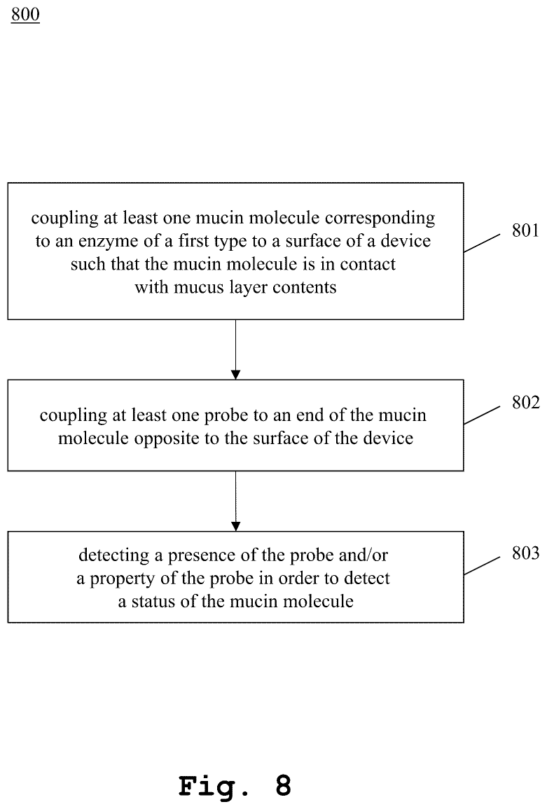

In FIG. 8, an exemplary embodiment of the method 800 according to the second aspect of the present disclosure is illustrated. In a first step 801, at least one mucin molecule corresponding to an enzyme of a first type is coupled to a surface of a device such that the mucin molecule is in contact with mucus layer contents. In a second step 802, at least one probe is coupled to an end of the mucin molecule opposite to the surface of the device. In a third step 803, a presence of the probe and/or a property of the probe are detected in order to detect a status of the mucin molecule.

Therefore, the present disclosure facilitates an in situ enzymatic activity detection scheme using mucin molecules attached to the external surface of the detector, which may measure the presence of enzymes produced by commensal bacteria residing in the mucus layer, e.g., in the mucus layer covering the cellular walls of GI-tract. The detector may continuously measure the degradation or removal of the mucin molecules due to digestion by the enzymes, e.g., the enzymes present inside the GI-tract.

Furthermore, by using multiple different mucin molecules attached to the external surface of the detector, a measurement on the presence and the activity of different enzymes can be provided, e.g., different enzymes produced by commensal bacteria residing inside the mucus layer of the GI-tract while the device may travel through the GI-tract. This may provide information on the health of mucus layer as well as a specific enzymatic signature, e.g., of the GI-tract.

It is important to note that, in the description as well as in the claims, the word “comprising” does not exclude other elements or steps and the indefinite article “a” or “an” does not exclude a plurality. A single element or other unit may fulfill the functions of several entities or items recited in the claims. Furthermore, the word “coupled” or “attached” implies that the elements may be directly connected together or may be coupled through one or more intervening elements. Moreover, the disclosure with regard to any of the aspects is also relevant with regard to the other aspects of the disclosure.

Although the present disclosure has been illustrated and described with respect to one or more implementations, equivalent alterations and modifications will occur to others skilled in the art upon the reading and understanding of this specification and the annexed drawings. In addition, while a particular feature of the present disclosure may have been disclosed with respect to only one of several implementations, such feature may be combined with one or more other features of the other implementations as may be desired for any given or particular application.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A device for detecting enzymatic activity in mucus layer, wherein the device comprises:
at least one mucin molecule corresponding to an enzyme of a first type, wherein the mucin molecule is coupled to a surface of the device and is configured to be in contact with mucus layer contents;
at least one probe coupled to an end of the mucin molecule opposite to the surface of the device; and
a detector configured to detect a presence of the probe and/or a property of the probe in order to detect a status of the mucin molecule.

2. The device of claim 1, wherein the mucin molecule comprises engineered human TR-O glycodomains or mucin-like domains decorated with O-glycans.

3. The device of claim 2, wherein the mucin molecule is configured to be digested by the enzyme of the first type produced by commensal microbes that reside in the mucus layer.

4. The device of claim 1, wherein the probe is an optical probe and the detector is configured to detect an optical signal from the probe, optionally, wherein the optical probe is a fluorescent probe and the detector is configured to detect a fluorescence of the fluorescent probe.

5. The device of claim 1, wherein the probe is a redox probe and the detector is configured to detect an oxidation-reduction activity of the redox probe.

6. The device of claim 1, wherein the device further comprises a linker molecule configured to couple the mucin molecule to the surface of the device, wherein the linker molecule is inert to the mucus layer contents and other gastrointestinal, GI, tract contents.

7. The device of claim 1, wherein the device further comprises:
at least one further mucin molecule corresponding to an enzyme of a second type, wherein the at least one further mucin molecule is coupled to the surface of the device and is configured to be in contact with the mucus layer contents; and
at least one further probe coupled to an end of the at least one further mucin molecule opposite to the surface of the device,
wherein the detector is configured to detect a presence of the further probe and/or a property of the further probe in order to detect a status of the at least one further mucin molecule.

8. The device of claim 7, wherein the at least one further mucin molecule is configured to be digested by the enzyme of the second type produced by commensal microbes that reside in the mucus layer.

9. The device of claim 7, wherein the detector is configured to detect the presence of the probe and the presence of the further probe simultaneously, or
wherein the detector is configured to detect the presence of the probe and the presence of the further probe sequentially, and/or
wherein the detector is configured to detect the property of the probe and the property of the further probe simultaneously, or
wherein the detector is configured to detect the property of the probe and the property of the further probe sequentially.

10. The device of claim 7, wherein the mucin molecule and the at least one further mucin molecule are coupled to the surface of the device at predefined locations, optionally in an array formation.

11. The device of claim 7, wherein the status of the mucin molecule or the at least one further mucin molecule is enzymatic degradation and/or removal of the mucin molecule or the at least one further mucin molecule.

12. A method for detecting enzymatic activity in mucus layer, the method comprises:
coupling at least one mucin molecule corresponding to an enzyme of a first type to a surface of a device such that the mucin molecule is in contact with mucus layer contents;
coupling at least one probe to an end of the mucin molecule opposite to the surface of the device; and
detecting a presence of the probe and/or a property of the probe in order to detect a status of the mucin molecule.

13. The method of claim 12, wherein the method further comprises:
coupling at least one further mucin molecule corresponding to an enzyme of a second type to the surface of the device such that the at least one further mucin molecule is in contact with the mucus layer contents;
coupling at least one further probe to an end of the at least one further mucin molecule opposite to the surface of the device; and
detecting a presence of the further probe and/or a property of the further probe in order to detect a status of the at least one further mucin molecule.

14. The method of claim 12, wherein the method further comprises:
detecting the presence of the probe and the presence of the further probe simultaneously, or
detecting the presence of the probe and the presence of the further probe sequentially, and/or
detecting the property of the probe and the property of the further probe simultaneously, or detecting the property of the probe and the property of the further probe sequentially.

15. The method of claim 13, wherein the status of the mucin molecule or the at least one further mucin molecule is enzymatic degradation and/or removal of the mucin molecule or the further mucin molecule.

16. A device for detecting enzymatic activity in a mucus layer, comprising:
a mucin molecule coupled to a surface of the device by a linker, wherein the mucin molecule is configured to be in contact with mucus layer contents;
at least one probe coupled to an end of the mucin molecule opposite to the surface of the device;
a detector configured to detect a signal from the probe; and
a transmitter configured to receive the signal from the probe and transmit the signal to an external device.

17. The device of claim 16, wherein the mucin molecule comprises engineered human TR-O glycodomains or mucin-like domains decorated with O-glycans.

18. The device of claim 17, wherein the mucin molecule is digestible by a glycosidase, a sulphatase, and/or a sialidase within the mucus layer.

19. The device of claim 18, wherein the probe is a fluorescent probe and the detector is a fluorescence detector.

20. The device of claim 18, wherein the probe is a redox probe and the detector is configured to detect an oxidation-reduction potential of the probe.
